(19) United States
(12) Patent Application Publication
(10) Pub. No.: US 2012/0065641 A1
(43) Pub. Date: Mar. 15, 2012

(54) METHODS AND DEVICES FOR REPAIRING BONE DEFECTS

(75) Inventors: Richard J. Deslauriers, Woodbury, CT (US); Eric Kolb, Sandy Hook, CT (US); John A. Tomich, Wallingford, CT (US); John Boxberger, Sandy Hook, CT (US); Naresh Akkarapaka, West Haven, CT (US)

(73) Assignee: Doctors Research Group, Inc., Southbury, CT (US)

(21) Appl. No.: 12/881,626

(22) Filed: Sep. 14, 2010

(51) Int. Cl. A61B 17/58; A61K 31/765; A61P 19/08; A61K 31/74

(52) U.S. Cl. 606/93; 424/78.08; 424/78.37

(57) ABSTRACT

A bone repair apparatus includes a partially polymerized biocompatible adhesive provided in a state wherein polymerization of the biocompatible adhesive is substantially suspended. A method for repairing a bone defect includes preparing the biocompatible adhesive, suspending polymerization of the biocompatible adhesive prior to full cure, delivering the biocompatible adhesive to the bone defect while polymerization is substantially suspended and accelerating polymerization of the biocompatible adhesive to achieve full cure. The method provides an implanted biocompatible adhesive with a malleable osteoconductive structure without compromising the physical characteristics of the biocompatible adhesive, thereby improving handling and delivery of the bone adhesive.

### FIELD OF THE INVENTION

The present invention relates to methods and devices for use in bone repair and, more particularly, to bone repair compositions and methods of implantation thereof.

### BACKGROUND OF THE INVENTION

There are many situations in which defects in bones or portions of bones must be repaired or replaced, including fractures, joint degeneration, abnormal bone growth, infection and the like. For instance, a bone fracture may result in a portion of missing bone that must be replaced. Similarly, an infection may result in the removal of a portion of bone also requiring replacement.

Conventional bone replacement technologies have developed a variety of bone defect fillers for use in orthopedic surgery to repair bones by filling bone voids, gaps, cracks and the like. These bone defect fillers may include a porous structure, which preferably has a high degree of pore interconnectivity, to provide an osteoconductive structure for bone ingrowth after the surgery. For instance, synthetic bone defect fillers, which are resorbable and porous, may replace bone with a bone-like mineral, e.g. crystalline hydroxyapatite or tricalcium phosphate. The resorbable and porous properties of these synthetic bone defect fillers allow for bone remodeling following implantation. However, conventional synthetic bone defect fillers are problematic because they have poor tensile, flexural, and shear properties and may adhere poorly to the surrounding bone, which can result in washout of the bone defect filler from the bone defect prior to ingrowth of new bone into the bone defect filler.

Conventional bone replacement technologies have also developed bone defect fillers with compositions that maintain their chemical and mechanical properties without change or subsequent remodeling (e.g., titanium, stainless steel, PMMA). However, these permanent bone defect fillers are problematic because, inter alia, they are not resorbable and/or cannot be molded and shaped for in situ curing.

Conventional bone replacement technologies have also developed bone defect fillers that may advantageously be shaped and/or molded in situ to conform to the shape of bone defects. For instance, bone defect fillers may be formed from collagen fiber structures that have fixed but pliable geometries. However, collagen fiber bone defect fillers, while shapeable, are problematic because they may have poor tensile and shear properties and may adhere poorly to the surrounding bone. Additionally, the degree of porosity provided by the collagen fiber structure may be adversely affected through the handling and application thereof such as during mixing of the material, when dispensed through a syringe or similar device, when packed into a surgical site or due to compression of by adjacent tissue or the like.

Other shapeable bone defect fillers have been formed from hydroxyapatite (HA) cement or tricalcium phosphate (TCP) cement. These bone defect fillers are initially flowable and then harden into a relatively rigid structure with an amorphous porosity on a micro scale. Additionally, like collagen fiber structures, the degree of porosity provided by cement bone defect fillers may be adversely affected through the handling and application thereof such as during mixing of the material, when dispensed through a syringe or similar device, when packed into a surgical site or due to compression by adjacent tissue or the like.

Biocompatible adhesives formed from polyurethane have also been developed as bone defect fillers for repairing bone defects. These polyurethane biocompatible adhesives may also advantageously be shaped and applied to the bone defect and allowed to cure in situ providing improved tensile strength and adhesive characteristics over other conventional bone defect fillers. Additionally, polyurethane biocompatible adhesives may be formed with a porous structure for promoting osteoconduction and new bone ingrowth. However, like shapeable bone defect fillers, the degree of porosity provided by polyurethane biocompatible adhesives may be adversely affected through the handling and application thereof such as during mixing of the material, when dispensed through a syringe or similar device, when packed into a surgical site or due to compression of by adjacent tissue or the like. Additionally, due to their improved adhesive characteristics, polyurethane adhesives may be difficult for a doctor to handle during polymerization thereof, since the polyurethane adhesives pass through a phase in which they are highly adhesive and may stick to unwanted surfaces.

To avoid adhesion to unwanted surfaces, established handling techniques for polyurethane adhesive include applying the polyurethane adhesive early or late to avoid the highly adhesive phase. However, this technique avoids optimal adhesion between the polyurethane adhesives and the surrounding bone since the polyurethane adhesive is not applied when most adhesive. Other established handling techniques include the use of instruments or disposable instruments. However, these techniques are also problematic since the former requires the polyurethane adhesive to reach a state of polymerization before instrument use or removal and the later operates under the expectation that the instrument will become coated in the polyurethane adhesive and ruined. A similar established handling technique requires that the user wear multiple pairs of gloves, which are removed as they become coated in the polyurethane adhesive. However, wearing and removing multiple pairs of gloves may be time consuming and, therefore, expensive for a surgeon to have to undertake during a surgical procedure. Additionally, this technique may be frustrating for the user and is wasteful, since it operates under the expectation that the gloves will become coated and in the polyurethane adhesive and ruined. Yet another established technique for handling polyurethane adhesive is to apply water or oil to surfaces contacting the polyurethane adhesive during handling to reduce local adhesion thereto. However, this technique is problematic in that it reduces adhesion of the polyurethane adhesive to its intended surfaces and may contaminate the polyurethane adhesive, which may have potentially adverse affects on the polymerization chemistry, such as increased expansion, decreased adhesive characteristics and/or decreased mechanical strength.

Accordingly, there is a need for a bone defect filler and a method of delivery thereof that provides a malleable osteoconductive structure without compromising the bone defect filler's physical and adhesive characteristics. Additionally, there is a need for a method of delivery of an in situ curing bone defect filler that improves handling thereof.

### SUMMARY OF THE INVENTION

According to the present invention, a method for repairing a bone defect includes preparing a biocompatible adhesive, suspending polymerization of the biocompatible adhesive prior to full cure, delivering the biocompatible adhesive to the bone defect while polymerization is substantially suspended and accelerating polymerization of the biocompatible adhesive to achieve full cure. Polymerization of the biocompatible adhesive may be effectively suspended over at least a three (3) month time period and, more preferably, over a two (2) year time period. The method provides an implanted bone defect filler with a malleable osteoconductive structure without compromising the bone adhesive's physical characteristics. Additionally, the method improves handling and delivery of the bone adhesive. Polymerization of the biocompatible adhesive may be substantially suspended by freezing the biocompatible adhesive.

According to some embodiments, the biocompatible adhesive is polyurethane adhesive. The biocompatible adhesive may be prepared by mixing a prepolymer component, a polyol component and a filler material. In some embodiments, the biocompatible adhesive transitions through an adhesive state during polymerization. In some embodiments of the method according to the present invention, the biocompatible adhesive may expand during polymerization.

According to some embodiments, the method may also include shaping the biocompatible adhesive into a rod, a sheet, a ball or the like prior to suspending polymerization. The biocompatible adhesive may be delivered with surgical gloves to the bone defect while polymerization is substantially suspended without the biocompatible adhesive substantially adhering to the surgical gloves.

According to the present invention, a bone repair apparatus includes a partially polymerized biocompatible adhesive provided in a state wherein polymerization of the biocompatible adhesive is substantially halted. The temperature of the biocompatible adhesive may is lowered to substantially halt polymerization of the biocompatible adhesive. In some embodiments, the temperature of the biocompatible adhesive is lowered to below −20° C. According to the present invention, polymerization of the biocompatible adhesive may be accelerated by elevating the temperature of the biocompatible adhesive.

According to some embodiments of the present invention, the biocompatible adhesive has a shape such as a rod, a sheet, a ball or the like. In some embodiments, the biocompatible adhesive is packaged in a cylindrical tube. In other embodiments, the biocompatible adhesive may be packaged in a low surface energy container. The biocompatible adhesive may also be packaged on a flexible sheet or the like.

According to the present invention, a bone repair product may be formed by a process including combining a prepolymer component, a polyol component and a filler material to form a mixture. The mixture is allowed to partially polymerize and then cooled to substantially halt polymerization thereof. The mixture may then be warmed to accelerate polymerization and allowed to fully polymerize. In some embodiments the mixture is a biocompatible polyurethane.

According to some embodiments, the mixture is partially polymerized to a target viscosity point, which may occur between four minutes and six minutes from initially combining the prepolymer component, polyol component and filler material.

In some embodiments, the mixture may be cooled to less then −20° C. to substantially halt polymerization thereof. The mixture may then be warmed to room temperature to accelerate polymerization. In other embodiments, the mixture is warmed to body temperature to accelerate polymerization.

According to the present invention, the bone repair product may also be formed by shaping the mixture. In some embodiments, the mixture is shaped by molding prior to cooling. In other embodiments, the mixture may be shaped by machining after cooling of the mixture.

These and other objects, features and advantages of the present invention will become apparent in light of the following detailed description of non-limiting embodiments, with reference to the accompanying drawings.

### BRIEF DESCRIPTION OF THE DRAWINGS

FIG. 1 is a perspective view of an bone defect filler proximate to a bone defect according to an embodiment of the present invention;

FIG. 2 is a method of preparing and implanting the bone defect filler of FIG. 1;

FIG. 3 is a perspective view of an embodiment for forming a biocompatible adhesive of the bone defect filler of FIG. 1;

FIG. 4 is a graph of an embodiment of a polymerization process of the biocompatible adhesive of FIG. 3;

FIG. 5 is a graph of another embodiment of the polymerization process of the biocompatible adhesive of FIG. 3;

FIG. 6 is yet another graph of an embodiment of the polymerization process of the biocompatible adhesive of FIG. 3;

FIG. 7 is a perspective view of an embodiment for partially polymerizing the biocompatible adhesive of FIG. 3;

FIG. 8 is a perspective view of the biocompatible adhesive of FIG. 3 formed to various shapes;

FIG. 9 is a perspective view of an embodiment for forming granules from the biocompatible adhesive of FIG. 3;

FIG. 10 is a perspective view of an embodiment for suspending polymerization of the biocompatible adhesive of FIG. 3;

FIG. 11 is a perspective view of an embodiment for packaging partially polymerized biocompatible adhesive according to the present invention;

FIG. 12 is a perspective view of implanted biocompatible adhesive according to an embodiment of the present invention;

FIG. 13 is a perspective view of an embodiment for unpackaging partially polymerized biocompatible adhesive according to the present invention;

FIG. 14 is a perspective view of another embodiment for implanting biocompatible adhesive according to the present invention;

FIG. 15 is a perspective view of partially implanted biocompatible adhesive of FIG. 14;

FIG. 16 is a perspective view of another embodiment for implanting biocompatible adhesive according to the present invention;

FIG. 17 is a perspective view of yet another embodiment of implanted partially polymerized biocompatible adhesive according to the present invention;

FIG. 18 is a perspective view of the implanted biocompatible adhesive of FIG. 17 after polymerization;

FIG. 19 is a perspective view of an embodiment of an interbody spacer having biocompatible adhesive disposed therein according to the present invention;

FIG. 20 is cross sectional view of the interbody spacer of FIG. 19 immediately after implantation;

FIG. 21 is a cross sectional view of the implanted interbody spacer of FIG. 20 after polymerization of the biocompatible adhesive disposed therein;

FIG. 22 is a perspective view of a bone filler device according to yet another embodiment of the present invention; and

FIG. 23 is a perspective view of another embodiment of a bone filler device according to the present invention.

### DETAILED DESCRIPTION OF A PREFERRED EMBODIMENT

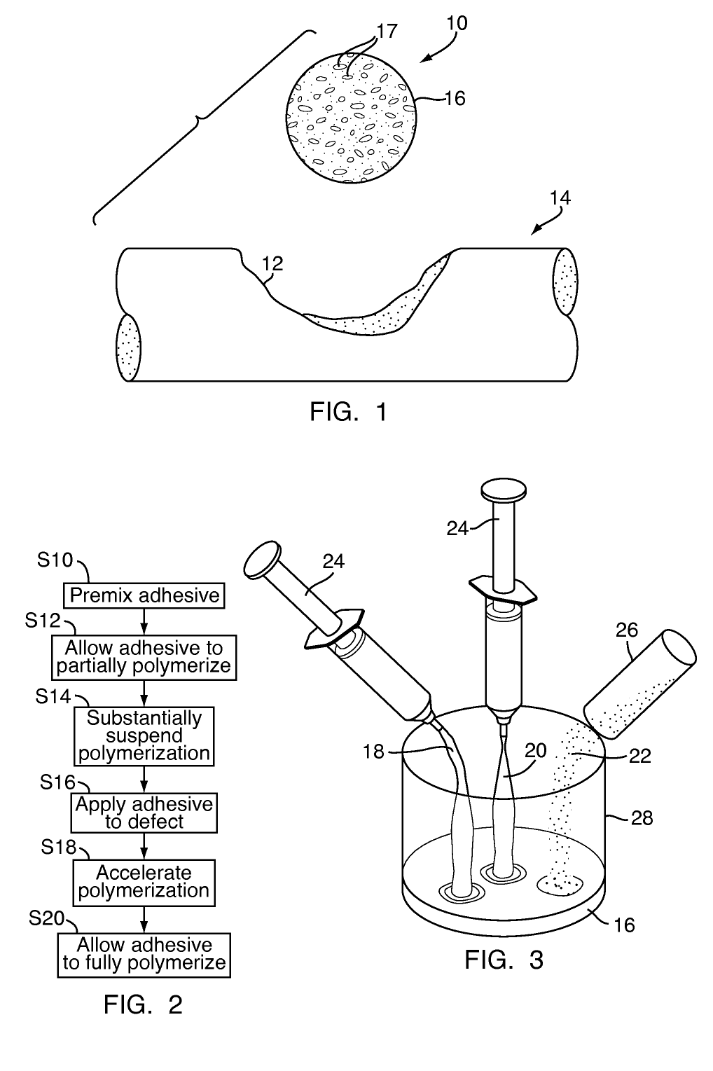

Referring to FIG. 1, a bone defect filler 10, for repairing a defect 12 in a bone 14 of a patient, includes a biocompatible adhesive 16 that is partially polymerized. The biocompatible adhesive 16 may have pores 17 partially formed therein during the partial polymerization, as will be discussed in greater detail below. Polymerization of the biocompatible adhesive 16 has been substantially suspended, as will also be discussed in greater detail below, to provide for improved handling of the bone defect filler 10, to reduce the likelihood of contamination of the bone defect filler 10 and to ease delivery of the bone defect filler 10 to the defect 12.

Referring to FIG. 2, to form the bone defect filler 10, shown in FIG. 1, the biocompatible adhesive 16, shown in FIG. 1, is initially mixed at step S10. Once mixed, the biocompatible adhesive 16, shown in FIG. 1, is allowed to partially polymerize at step S12. Polymerization of the biocompatible adhesive 16, shown in FIG. 1, is then substantially suspended at step S14. Polymerization of the biocompatible adhesive may be effectively suspended over at least a three (3) month time period and, more preferably, over a two (2) year time period. Once polymerization of the biocompatible adhesive 16, shown in FIG. 1, has been substantially suspended at step S14, the bone defect filler 10, shown in FIG. 1, is typically stored for future use, as will be discussed in greater detail below.

When ready for use, the bone defect filler 10, shown in FIG. 1, may then be applied to the defect 12, shown in FIG. 1, at step S16. Polymerization of the biocompatible adhesive 16, shown in FIG. 1, may then be accelerated at step S18. Once polymerization has been accelerated, the biocompatible adhesive 16, shown in FIG. 1, may be shaped in situ, if desired, and is allowed to fully polymerize within the defect 12, shown in FIG. 1, at step S20. When the biocompatible adhesive 16, shown in FIG. 1, has fully polymerized, the bone defect filler 10, shown in FIG. 1, fills the defect 12, shown in FIG. 1 and provides a strong osteoconductive structure that promotes bone ingrowth. In some embodiments, polymerization of the biocompatible adhesive 16, shown in FIG. 1, may be accelerated before the biocompatible adhesive 16, shown in FIG. 1, is applied to the defect 12, shown in FIG. 1. Acceleration prior to application may advantageously allow the biocompatible adhesive 16, shown in FIG. 1, to become malleable to allow some degree of contouring before application to the defect 12, shown in FIG. 1. For example, in some embodiments, polymerization of the biocompatible adhesive 16, shown in FIG. 1, may be accelerated simply during transport from storage to the application site.

Referring to FIG. 3, the biocompatible adhesive 16 mixed at step S10, shown in FIG. 2, may be a polyurethane adhesive formed by combining and mixing a prepolymer component 18 and a polyol component 20, along with an optional filler material 22, and permitting the combination to react to form the biocompatible adhesive 16. An example of one suitable polyurethane adhesive for the biocompatible adhesive 16 is the KRYPTONITE™ bone cement product, available from DOCTORS RESEARCH GROUP, INC. of Southbury, Conn., which is described in U.S. patent application Ser. No. 11/089,489, which is hereby incorporated by reference in its entirety.

In some embodiments, the prepolymer component 18, polyol component 20 and optional filler material 22, if included, may be provided as a kit for forming the biocompatible adhesive 16. In the kit, the prepolymer component 18, polyol component 20 and optional filler material 22 are each held in a suitable container such as syringes 24, canister 26 or the like until combined in a mixing container 28.

The prepolymer component 18 for forming the biocompatible adhesive 16 includes prepolymer molecules formed by reacting diisocyanate with polyol. The prepolymer component 18 may be a true prepolymer, formed with a two to one ratio of diisocyanate to polyol, or the prepolymer component 18 may be a quasi-prepolymer, formed with a ratio of diisocyanate to polyol in excess of two to one. As will be understood by those skilled in the art, a broad variety of diisocyanates and polyols may be suitable for use in the prepolymer component 18 and the biocompatible adhesive 16 of the present invention. Both aromatic and aliphatic diisocyanates may be used to form the prepolymer component 18 of the present invention. The polyol used to form the prepolymer component may be the same as or different than the polyol of the polyol component 20. Additionally, the polyol used to form the prepolymer component 18 may be a blend of different polyols to achieve desired properties. Various polyols suitable for synthesis with the diisocyanate will be discussed in greater detail below.

The polyol component 20 for forming the biocompatible adhesive 16 may include naturally occurring polyols and biocompatible, synthetic polyols, and mixtures thereof to achieve desired properties in the biocompatible adhesive 16. The polyol component 20 preferably also includes a catalyst for controlling and/or reducing the time required for polymerization of the biocompatible adhesive 16. Additionally, the polyol component 20 may include water, which is known to react with diisocyanate to produce carbon dioxide. Thus, the water may be provided to react with the diisocyanate to generate a sufficient amount of carbon dioxide to impart a degree of porosity to the biocompatible adhesive 16, as will be discussed in greater detail below. Alternatively, rather than including water in the polyol component 20, moisture from the atmosphere or moisture included in the optional filler material 22 may impart the degree of porosity to the biocompatible adhesive 16. Additionally, in instances where moisture is provided from the atmosphere or within the optional filler material 22, it may be desirable to dry the polyols to provide improved control over the amount of carbon dioxide produced and, therefore, the degree of porosity imparted to the biocompatible adhesive 16.

The optional filler material 22 for forming the biocompatible adhesive 16 may include, but is not limited to, calcium carbonate, bone (e.g., demineralized bone, allograft bone, and/or autogenous bone), calcium phosphate, calcium pyrophosphate, hydroxyapatite, poly methyl methacrylate, glass-ionomer, calcium sulfate, tricalcium phosphate (e.g., beta tricalcium phosphate), or any combination thereof, or the like. In certain embodiments, the filler material 22 may be chosen so as to form pores 17, shown in FIG. 1, within the biocompatible adhesive 16 to impart a desired degree of porosity to the biocompatible adhesive 16. For example, the filler material 22 may include water for reacting with the diisocyanate of the prepolymer component 18 to generate carbon dioxide and impart the porosity to the biocompatible adhesive 16. The filler material 22 may also be present in the biocompatible adhesive 16 in an amount sufficient to modify the biocompatible adhesive's mechanical properties (e.g., compressive strength, compressive modulus, Young's Modulus of Elasticity, flexural strength, and the like). The filler material 22 may also comprise calcium carbonate and, in certain of these embodiments, the filler material 22 may comprise calcium carbonate in an amount sufficient to provide free calcium to a body of a mammal and enhance osteoconductivity and to provide porosity once the calcium carbonate has been resorbed or migrated from the implantation site. In some embodiments, the optional filler material 22 may include barium sulfate, calcium phosphate, bone graft, antibiotics, osteoinductive agents, or the like to be delivered to the bone defect 12, shown in FIG. 1, through implantation of the biocompatible adhesive 16 at step S16, shown in FIG. 2.

Although the biocompatible adhesive 16 may be formed with a variety of compositions to achieve desired properties, preferably, the prepolymer component 18 includes aromatic pMDI diisocyanates synthesized with polyols derived from castor oil. The polyol component 20 preferably also includes polyols derived from castor oil and a small percentage of tin based acid as a catalyst. The optional filler material 22 is preferably calcium carbonate powder, at a concentration of thirty percent (30%) by weight, with approximately ninety percent (90%) of the powdered particles being less than ten microns (10 μm) in diameter.

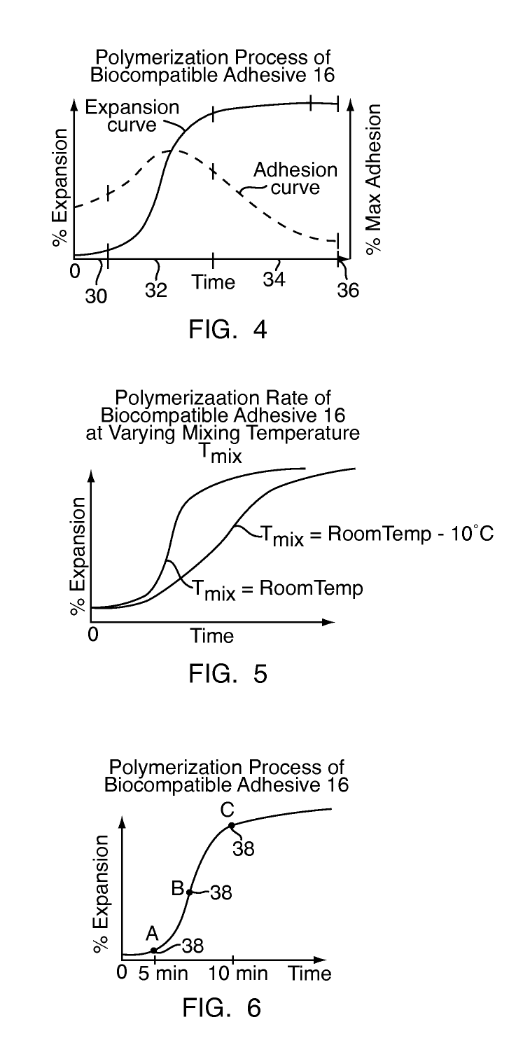

Referring to FIG. 4, the polymerization process of the biocompatible adhesive 16 is shown at room temperature. Initially, when the prepolymer component 18, polyol component 20 and optional filler material 22 are combined and mixed, the biocompatible adhesive 16 is in a liquid state 30. The biocompatible adhesive 16 then passes through a taffy-like state 32 in which the biocompatible adhesive 16 expands, is highly adhesive and easily malleable. Then, the biocompatible adhesive 16 passes through a putty-like state 34 in which expansion of the biocompatible adhesive 16 slows and the biocompatible adhesive 16 becomes less adhesive yet is still malleable. Finally, the biocompatible adhesive 16 cures into a solid state 36. During the polymerization process and, particularly during the taffy-like state 32, carbon dioxide byproducts of the chemical reaction cause expansion of the biocompatible adhesive 16 and form pores 17, shown in FIG. 1, within the biocompatible adhesive 16. Although the polymerization process has been shown in FIG. 4 for the exemplary biocompatible adhesive 16 discussed above, one skilled in the art should understand that variations in the chemical composition of the biocompatible adhesive 16 may increase or decrease the timing and/or magnitude of expansion and polymerization.

Referring back to FIG. 3, small quantities of the biocompatible adhesive 16 may be formed by mixing the prepolymer component 18, polyol component 20 and optional filler material 22 by hand. However, since hand mixing may be problematic for larger bulk quantities, bulk quantities may instead be mixed by a mechanical mixing device (not shown). It is important that the biocompatible adhesive 16 achieve a homogeneous solution through mixing so that the biocompatible adhesive 16 has uniform properties and so that, if bulk quantities are partitioned into multiple bone defect fillers 10, shown in FIG. 1, all partitioned bone defect fillers 10, shown in FIG. 1, have equivalent contents and properties. Preferably, mixing of the prepolymer component 18, polyol component 20 and optional filler material 22 is carried out at relatively low speeds so as not to fully disperse small clumps of the optional filler material 22, which act as nucleation sites for generation of pores 17, shown in FIG. 1. For example, mixing may be performed at a speed consistent with manual mixing with a fixed spatula or the like. Preferably, after mixing, these small clumps of filler material 22 are sufficiently sized to generate an average pore size for pores 17, shown in FIG. 1, within the bone defect filler 10, shown in FIG. 1, within the ideal range of one hundred micrometers (100 μm) to five hundred micrometers (500 μm) for allowing bone ingrowth. In other embodiments, where pore size may be less critical, high speed mixing may be implemented to fully disperse the small clumps of filler material 22, spreading them evenly within the biocompatible adhesive 16 and reducing the opportunity for these clumps to act as nucleation sites for generation of pores 17, shown in FIG. 1.

Referring to FIG. 5, the mixing step S10, shown in FIG. 2, may also include controlling a mixing temperature Tₘᵢₓ of the biocompatible adhesive 16 during the mixing step S10, shown in FIG. 2. Controlling the mixing temperature Tₘᵢₓ may alter the polymerization rate of the biocompatible adhesive 16. For example, the polymerization rate of the exemplary biocompatible adhesive 16 is approximately halved for every ten degrees Celsius (10° C.) of temperature reduction. Thus, reducing the mixing temperature Tₘᵢₓ may advantageously allow additional time to mix the biocompatible adhesive 16 in bulk and to partition the biocompatible adhesive 16 into smaller portions before substantial polymerization has occurred. In some embodiments, the mixing temperature Tₘᵢₓ may be reduced by conducting the mixing step S10, shown in FIG. 2, in a cool or cold room or, in other embodiments, the mixing container 28, shown in FIG. 3, may be a temperature controlled container for providing the cool or cold mixing environment for reducing the mixing temperature Tₘᵢₓ.

Referring to FIG. 6, once the mixing step S10, shown in FIG. 2, is complete, the biocompatible adhesive 16 is allowed to partially polymerize at step S12, shown in FIG. 2, until the biocompatible adhesive 16 reaches a target viscosity point 38, at which point polymerization of the biocompatible adhesive 16 is substantially suspended at step S14, shown in FIG. 2. The target viscosity point 38 may be selected at essentially any time during the polymerization process depending upon the desired application for the biocompatible adhesive 16. For example, the target viscosity point 38 may be selected to be before, at or after gelation of the biocompatible adhesive 16.

For instance, for some applications, the target viscosity point 38 may be selected at point C, where substantially all of the expansion of the biocompatible adhesive 16 has occurred. Applications that may benefit from suspension of polymerization after substantially all expansion of the biocompatible adhesive 16 may include those where in situ expansion would cause risk to the patient, e.g. close proximity to neural structures or internal organs, or applications that could result in a physical deformity if expansion occurs, such as cranial defect repairs. While point C may be desirable for applications where minimal expansion is desired, the biocompatible adhesive 16 also has reduced adhesion characteristics at point C since many of the reactive isocyanate groups of the prepolymer component 18, shown in FIG. 3, have already been consumed during polymerization and are, therefore, unavailable for adhesion to bone 14, shown in FIG. 1, after implantation in the bone defect 12, shown in FIG. 1. Additionally, at point C, the biocompatible adhesive 16 may be overly viscous so as not to fully wet the surface of the bone 14, shown in FIG. 1, within the bone defect 12, shown in FIG. 1, which may result in less than maximum adhesion for the bone defect filler 10, shown in FIG. 1.

Still referring to FIG. 6, for some applications of the bone defect filler 10, shown in FIG. 1, the target viscosity point 38 may instead be selected at point A, which is prior to substantial expansion of the biocompatible adhesive 16. At point A, the biocompatible adhesive 16 is still substantially in the liquid state 30, shown in FIG. 4 and has not yet reached its maximum adhesive potential. Selecting point A for as the target viscosity point 38 may be desirable where adhesion to the bone 14, shown in FIG. 1, is critically important, e.g. for repair of fractures or the like, and/or where in situ expansion of the biocompatible adhesive 16 may be desirable to drive the biocompatible adhesive 16 into porous bone 14, shown in FIG. 1, to enhance fixation thereto.

Alternatively, rather than setting the target viscosity point 38 as point A, prior to substantially any expansion, or point C, after substantially all expansion, in some embodiments, the target viscosity point 38 may be selected as point B, which occurs at some time between points A and C after partial expansion of the biocompatible adhesive 16. Selecting point B as the target viscosity point 38 may achieve an optimum balance of adhesive potential and expanded state for the biocompatible adhesive 16 when polymerization is substantially suspended in step S14, shown in FIG. 2.

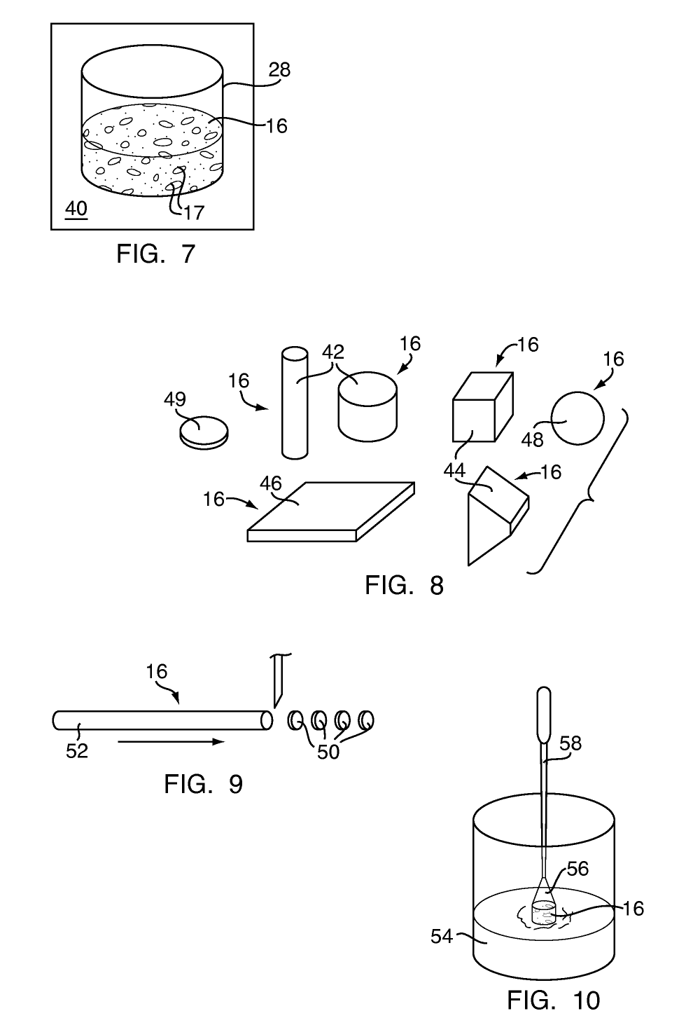

Referring to FIG. 7, as discussed above, carbon dioxide may be generated during polymerization of the biocompatible adhesive 16, which forms pores 17 within the biocompatible adhesive 16 and imparts porosity to the biocompatible adhesive 16. In some embodiments of the present invention, the partial polymerization step S12 may be conducted in a controlled environment 40 to control the porosity imparted to the biocompatible adhesive 16 prior to suspension of polymerization in step S14, shown in FIG. 2. For example, the controlled environment 40 may be a vacuum environment, e.g. a reduced pressure environment, which increases the degree of porosity within the biocompatible adhesive 16 by allowing the entrapped carbon dioxide to expand more than under atmospheric conditions, thereby forming larger pores 17.

Referring to FIG. 8, in some embodiments of the present invention, the biocompatible adhesive 16 may also be formed to various shapes and/or sizes during the partial polymerization step S12, shown in FIG. 2, to tailor the bone defect filler 10, shown in FIG. 1, to a particular application thereof. For example, the biocompatible adhesive 16 may be formed in rods 42, blocks 44, sheets 46, balls 48, disks 49 or the like. The biocompatible adhesive 16 may be formed into these various shapes and sizes by molding, pouring, stretching, rolling, through hand manipulation or the like. Additionally, in some embodiments, the biocompatible adhesive 16 may be formed in bulk and partitioned into a plurality of smaller portions that may be substantially uniform in size and shape or, alternatively, may be of varying sizes and shapes.

Referring to FIG. 9, in some embodiments, the biocompatible adhesive 16 may be formed into a plurality of beads or granules 50 during the partial polymerization step S12, shown in FIG. 2. For example, the biocompatible adhesive 16 may be formed as a thin member 52 and then cut or sliced to form the beads or granules 50, which may be formed in a variety of particle sizes and shapes. In other embodiments, the granules 50 may be formed by dispensing the biocompatible adhesive 16 from a dispenser (not shown) at a desired particle size.

Referring back to FIG. 2, once the biocompatible adhesive 16, shown in FIG. 3, has polymerized to the desired target viscosity point 38, shown in FIG. 6, polymerization of the biocompatible adhesive 16, shown in FIG. 3, is substantially suspended at step S14. For example, polymerization may be substantially suspended by reducing the temperature of the biocompatible adhesive 16 to below its freezing point, which may be in the range of negative twenty degrees Celsius (−20° C.) to negative eighty degrees Celsius (−80° C.) for polyurethane adhesives such as the exemplary biocompatible adhesive 16 described above. Additionally, those skilled in the art should understand that modification of the chemical formulation of the biocompatible adhesive 16, shown in FIG. 3, may alter the freezing point such that the chemical formulation may be manipulated to place the freezing point above or below the commonly available storage temperatures in hospitals.

Referring to FIG. 10, in some embodiments, freezing of the biocompatible adhesive 16 may be achieved by rapid cooling in liquid nitrogen 54. In these embodiments, the partially polymerized biocompatible adhesive 16 is submerged in liquid nitrogen 54, which freezes the biocompatible adhesive 16 and suspends polymerization thereof. Prior to submerging the biocompatible adhesive 16, the biocompatible adhesive 16 is preferably enclosed within a protective container 56, e.g. a device or packaging to retain the shape of the biocompatible adhesive 16 and to prevent direct contact between the liquid nitrogen 54 and the biocompatible adhesive 16. The protective container 56 with the biocompatible adhesive 16 therein may then be lowered into the liquid nitrogen 54 with a submersion tool 58 to suspend polymerization of the biocompatible adhesive 16. Advantageously, the time to freeze the biocompatible adhesive 16 with the liquid nitrogen 54 is only on the order of seconds.

In other embodiments, freezing of the biocompatible adhesive 16 may be achieved through cooling in a freezer (not shown). For example, commercial freezers (not shown) are readily available at temperatures within the range of negative forty degrees Celsius (−40° C.) to negative eighty degrees Celsius (−80° C.). In these embodiments, the biocompatible adhesive 16 may be placed in the freezer (not shown) immediately upon mixing in step S10, shown in FIG. 2, to suspend polymerization of the biocompatible adhesive 16 within minutes. The rate that polymerization is substantially suspended within the freezer (not shown) will be dependent on a number of factors including the volume of the biocompatible adhesive 16, the temperature of the freezer (not shown) and the heat transfer coefficient between the biocompatible adhesive 16 and the cool environment within the freezer (not shown) and, therefore, the rate of substantially suspended polymerization may be controlled by varying these factors.

Once the biocompatible adhesive 16 has been frozen to suspend polymerization thereof, it is preferably stored and transported in the frozen state to maintain the substantially suspended polymerization until the biocompatible adhesive 16 is needed in orthopedic surgery, for example, to repair of a bone defect 12, shown in FIG. 1, or the like. The frozen biocompatible adhesive 16 will see a significant increase in stiffness relative to biocompatible adhesive 16 in the liquid, taffy or putty-like stages of polymerization at room temperature, which may be highly beneficial for transport and handling thereof. Transport of the biocompatible adhesive 16 may involve overnight delivery with the biocompatible adhesive 16 packaged in dry ice (not shown) to keep the temperature sufficiently cold to maintain substantially suspended polymerization. When not in transport, the biocompatible adhesive 16 may be stored in an appropriate freezer (not shown), to maintain substantially suspended polymerization thereof. Preferably, at a hospital, the freezer (not shown) is located in or near the operating facilities within the hospital. Failure to maintain the biocompatible adhesive 16 at an appropriate temperature to maintain substantially suspended polymerization will result in polymerization continuing, which may decrease a shelf-life of the bone defect filler 10 or render it unusable. Therefore the biocompatible adhesive 16 may be packaged and transported with a temperature alarm (not shown) that monitors the biocompatible adhesive's temperature and generates a notification, e.g. an audio alert, an alert message, a warning light, a colored indicator or the like, if the appropriate temperature is not maintained.

Although storage of the bone defect filler 10 comprising frozen biocompatible adhesive 16 in substantially suspended polymerization has been described above as occurring below the freezing point of the biocompatible adhesive 16, in some embodiments, it may be preferable to store the bone defect filler 10 just above the freezing point of the biocompatible adhesive 16. Storing the bone defect filler 10 at a temperature just above the freezing point of the biocompatible adhesive 16 will substantially slow the polymerization process without eliciting any phase change that could adversely affect the structure of polymer chains of the biocompatible adhesive 16. Additionally, the stiffness of the biocompatible adhesive 16 when stored at just above its freezing point will still be far greater than at room temperature and, therefore, will still provide improved handling and transportation characteristics.

Referring back to FIG. 8, although shaping of the biocompatible adhesive 16 was discussed above in connection with the partial polymerization step S12, shown in FIG. 2, shaping of the biocompatible adhesive 16 may also be conducted after polymerization of the biocompatible adhesive 16 has been substantially suspended in step S14, shown in FIG. 2. For example, the biocompatible adhesive 16 may be machined, e.g. by cutting, slicing, grinding or the like, while in substantially suspended polymerization to form the rods 42, blocks 44, sheets 46, balls 48 or other desired shapes. Similarly, referring to FIG. 9, the granules 50 may be formed by first suspending polymerization of the biocompatible adhesive 16 and then cutting or grinding the biocompatible adhesive 16 into granules 50.

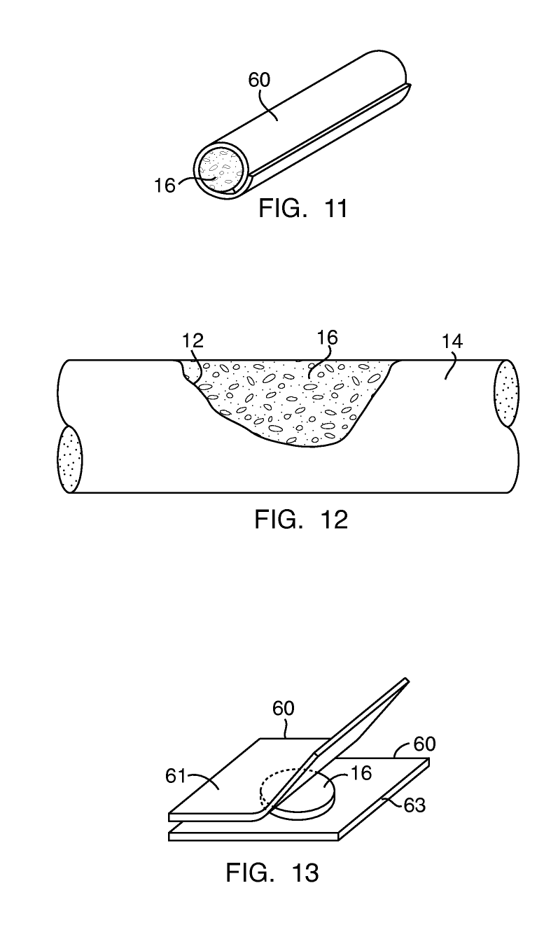

In addition to the suspension of polymerization and material processing, step S14 may also include sterilization, packaging and labeling of the biocompatible adhesive 16. For example, referring to FIG. 11, the biocompatible adhesive 16 may be packaged in a low surface energy material 60, such as medical grade silicone sheeting, to allow complete and easy removal of the biocompatible adhesive 16 from the packaging. In some embodiments, the biocompatible adhesive 16 may first be packaged in the low surface energy material 60 and then packaged in a secondary outer layer (not shown) that is more rigid to maintain the structure of the biocompatible adhesive 16, for example, when the biocompatible adhesive 16 is to be stored at a temperature greater than its freezing point. Additionally, the secondary outer layer (not shown) may provide a sterile environment for the biocompatible adhesive 16 to protect the biocompatible adhesive 16 from contaminants or the like. In some embodiments the biocompatible adhesive 16 may be sandwiched between two low surface energy sheets 60 and, yet in other embodiments, the biocompatible adhesive 16 may be packaged in a low surface energy sheet 60 formed as a bag designed to allow the biocompatible adhesive 16 to be dispensed through a single small opening by squeezing or rolling the low surface energy sheet 60 as polymerization of the biocompatible adhesive 16 within the bag accelerates. In these embodiments, the packaged biocompatible adhesive 16 is preferably also contained within an additional sterile container (not shown), such as a sterile bag or box.

As discussed above, the biocompatible adhesive 16 with polymerization substantially suspended, is shipped to the hospital under temperature control and likely stored in a freezer (not shown) near the operating room. Referring back to FIG. 2, during a surgical procedure, the surgeon will determine when the biocompatible adhesive 16 is needed to be applied to the bone defect 12, shown in FIG. 1, at step S16. At the appropriate time, the surgeon, therefore, requests that the biocompatible adhesive 16, shown in FIG. 1, be transferred to the operating room.

Referring to FIG. 8, as discussed above, the biocompatible adhesive 16 may be formed and stored in various shapes and sizes. Thus, the surgeon may advantageously select an appropriately sized and/or shaped specimen of frozen biocompatible adhesive 16 to fill the defect 12, shown in FIG. 1. The varying sizes and shapes allow the surgeon to select an appropriate size and/or shape for a broad range of clinical applications. For example rods 42 may be selected to fill bone defects 12, shown in FIG. 1, in the form of holes formed by the removal of screws (not shown) or pins (not shown) from within the bone 14, shown in FIG. 1. The rod 42 may also advantageously be inserted within a hole in the bone 14 to augment implantation of a new screw (not shown) or pin (not shown) by allowing the screw (not shown) or pin (not shown) to be implanted within the rod 42 during or after full polymerization thereof. As will be appreciated by those skilled in the art, rods 42 of varying sizes may also be desirable for use during interbody fusion, sternotomy repair, facet fusion, cranial repair or the like. Biocompatible adhesive 16 formed as blocks 44 may be advantageously implanted as interbody spacers or to repair the iliac crest following osteotomy. Sheets 46 of biocompatible adhesive 16 may be advantageous for use during spinal reconstruction and/or laminoplasty. Additionally, the sheet 46 may be particularly useful in that in situ forming of a sheet shape from a non-frozen adhesive is particularly challenging. Granules 50, shown in FIG. 9, and/or balls 16 may be advantageous for repair of irregular internal bone defects 12, shown in FIG. 1, such as cysts or the like. Additionally, rather than selecting biocompatible adhesive 16 of a particular size and/or shape based on intended application, the surgeon may also advantageously select an oversized shape that may be cut to an appropriate size interoperatively prior to implantation thereof. In some embodiments, cutting of the biocompatible adhesive 16 may occur through the packaging material, such as the low surface energy sheet 60, shown in FIG. 11, to minimize direct contact with the biocompatible adhesive 16.

Referring back to FIG. 1, the biocompatible adhesive 16 is preferably transported to maintain the biocompatible adhesive 16 at a cool temperature during transport from the freezer (not shown) and in the final moments before application of the biocompatible adhesive 16 to the bone defect 12. For example, the biocompatible adhesive 16 may be transported in an insulated container (not shown). Alternately, the biocompatible adhesive 16 may be transported with a conductive structure (not shown), such as a cold piece of metal, in close proximity to the packaged biocompatible adhesive 16, shown in FIG. 11, that acts to draw heat from the biocompatible adhesive 16.

Prior to application of the biocompatible adhesive 16 to the defect 12, the surgical technique may necessitate that the surgeon clean and dry the surface of the bone 14, within the defect 12 in an effort to maximize the potential for adhesion of the biocompatible adhesive 16 to the bone 14.

Referring to FIG. 12, the biocompatible adhesive 16 may then be applied to the bone defect 12 manually or using an application instrument. In some embodiments, it may be desirable to apply the biocompatible adhesive 16 to the defect 12 immediately while still in the cool/frozen state with substantially suspended polymerization, while in other embodiments, it may be desirable to accelerate polymerization and allow the biocompatible adhesive 16 to at least partially thaw prior to implantation within the defect 12. As the biocompatible adhesive 16 thaws and softens, it is possible to contour the biocompatible adhesive 16 to maximize fit within the defect 12. Thus, allowing the biocompatible adhesive 16 to thaw prior to implantation may allow the surgeon to shape and contour the biocompatible adhesive 16 to better match the shape of the defect 12. For example, the surgeon may contour the sheet 46 to match the shape of a cranium for cranial repair. In some embodiments, accelerating polymerization of the biocompatible adhesive 16 prior to implantation may allow the surgeon to push the biocompatible adhesive 16 into the defect 12 during implantation to achieve strong contact with the bone 14 and partially drive the biocompatible adhesive 16 into the bone 14. In some embodiments of the present invention, the biocompatible adhesive 16 may be contoured and/or smoothed after application to the defect 12. Preferable, contouring, shaping and/or smoothing of the biocompatible adhesive 16 is conducted within or through at least a portion of the packaging, e.g. the low surface energy sheet 60 shown in FIG. 11, to minimize direct contact with the biocompatible adhesive 16.

The temperature of the biocompatible adhesive 16 rises when the biocompatible adhesive 16 is exposed to room temperature in the operating room and/or the patient's body temperature when implanted in the defect 12. As the temperature of the biocompatible adhesive 16 rises, polymerization of the biocompatible adhesive 16 accelerates at step S18, shown in FIG. 2.

Still referring to FIG. 12, the chemical formulation, volume and shape of the biocompatible adhesive 16, as well as the local heat transfer properties, e.g. due to different packaging materials, will govern how quickly the biocompatible adhesive 16 warms and accelerates polymerization. For example, in some embodiments, the biocompatible adhesive 16 may warm within seconds or minutes and reach adequate in situ polymerization by the end of the surgical procedure. In embodiments where the biocompatible adhesive 16 is allowed to partially thaw/warm prior to implantation within the defect 12 it may be beneficial to deliver the biocompatible adhesive 16 before it reaches a temperature at which it is tacky and relatively difficult to handle. As should be understood by those skilled in the art, for many applications there may be an optimum time of application where the biocompatible adhesive 16 is sufficiently thawed yet not so tacky as to limit handling thereon. In particular, it may be advantageous to peel packaging away from the biocompatible adhesive 16 while polymerization thereof is substantially suspended, thereby allowing the biocompatible adhesive 16 to maintain its shape. For example, referring to FIG. 13, as discussed above, the biocompatible adhesive 16 may be packaged between two low surface energy sheets 60, a first sheet 61 and a second sheet 63. Once the biocompatible adhesive 16 has been delivered to the operating room between the two low surface energy sheets 60, the first sheet 61 may be peeled away from the biocompatible adhesive 16 while polymerization is still substantially suspended, without damaging or altering the shape of the biocompatible adhesive 16. The second sheet 63 may then be used to deliver the biocompatible adhesive 16 to the defect 12, shown in FIG. 12, and contour the biocompatible adhesive 16, if desired. Preferably, the second sheet 63 is then also peeled away from the biocompatible adhesive 16 in substantially the same manner as the first sheet 61 before the biocompatible adhesive 16 becomes tacky and difficult to handle.

Since the process of thawing the biocompatible adhesive 16 may lead to moisture condensation on the surface of the biocompatible adhesive 16, which, in the case of a polyurethane adhesive, for example, may act as a contaminant to the chemical reaction, in some embodiments, the biocompatible adhesive 16 may be thawed within its packaging, such that moisture condenses on the packaging, not the adhesive material itself. Alternatively, in other embodiments, the biocompatible adhesive 16 may be delivered directly to the defect 12, placing the biocompatible adhesive 16 in direct contact with bone 14 within the defect 12, thereby reducing exposure of the portions of the biocompatible adhesive 16 that contact the bone 14 to the external environment and the undesirable effects of condensation.

In some embodiments, external heat sources (not shown), such as heat lamps, may additionally be applied to the biocompatible adhesive 16 to speed thawing/warming. Advantageously, these heat sources (not shown) may also elevate the temperature of the biocompatible adhesive 16 above room temperature to increase the polymerization rate thereof.

Once polymerization of the biocompatible adhesive 16 has accelerated, the biocompatible adhesive 16 is allowed to fully polymerize at step S20, shown in FIG. 2. The time until the biocompatible adhesive 16 is fully polymerized may be in excess of twenty-four (24) hours and may depend upon the chemical formulation of the biocompatible adhesive 16. Additionally, depending upon the state at which polymerization of the biocompatible adhesive 16 was substantially suspended, as discussed above in connection with FIG. 6, some expansion of the biocompatible adhesive 16 may occur during the remainder of the polymerization process. Advantageously, this expansion may drive the biocompatible adhesive 16 into local porous bone (not shown) of the bone 14.

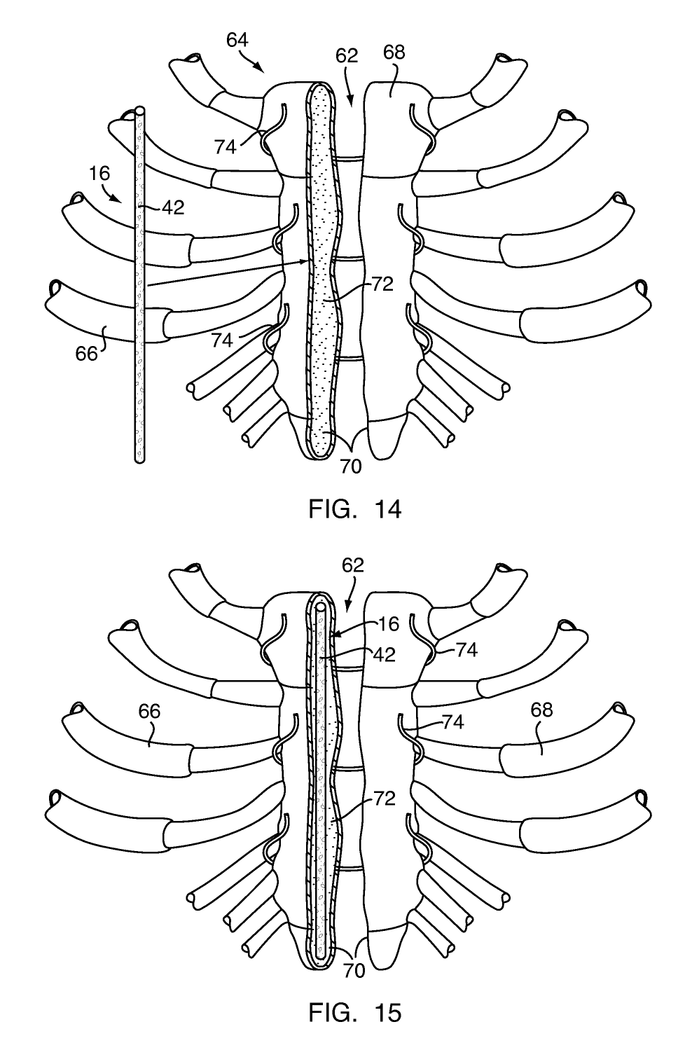

Referring to FIG. 14, in some embodiments, the biocompatible adhesive 16 shaped as rod 42 may advantageously be implemented for sternotomy repair, as discussed above. For example, during a sternotomy procedure, an incision 62 is made in a sternum 64 to separate the sternum 64 into at least a first sternum portion 66 and a second sternum portion 68. Each of the first and second sternum portions 66 and 68 includes a cut surface 70 at the incision 62 exposing cancellous bone 72 within the sternum 64. At the end of the sternotomy procedure, the frozen rod 42 of biocompatible adhesive 16 may be applied to the cut bone surfaces 70 to enhance adhesion and closure therebetween.

Referring to FIG. 15, preferably, the rod 42 approximates the length of the sternum 64 or, alternately, the rod 42 may be cut or provided as multiple shorter segments. The frozen rod 42 is partially packed into the cancellous bone 72 of one of the first or second sternum portions 66 or 68. Then, wires 74, or similar closure devices, are tightened to approximate the first and second sternum portions 66 and 68, forcing the biocompatible adhesive 16 onto and into the opposing sternum portion 66 or 68. Subsequent heating of the biocompatible adhesive 16, for example, from the patient's body heat or another heating device accelerates polymerization and initiates expansion of the biocompatible adhesive 16 into the cancellous bone 72. This embodiment advantageously provides for expansion of the biocompatible adhesive 16 into the first and second sternum portions 66 and 68 through a mess free application relative to application of the biocompatible adhesive 16 in the liquid state 30, shown in FIG. 4, or the taffy state 32, shown in FIG. 4, using a spatula (not shown) or syringe (not shown).

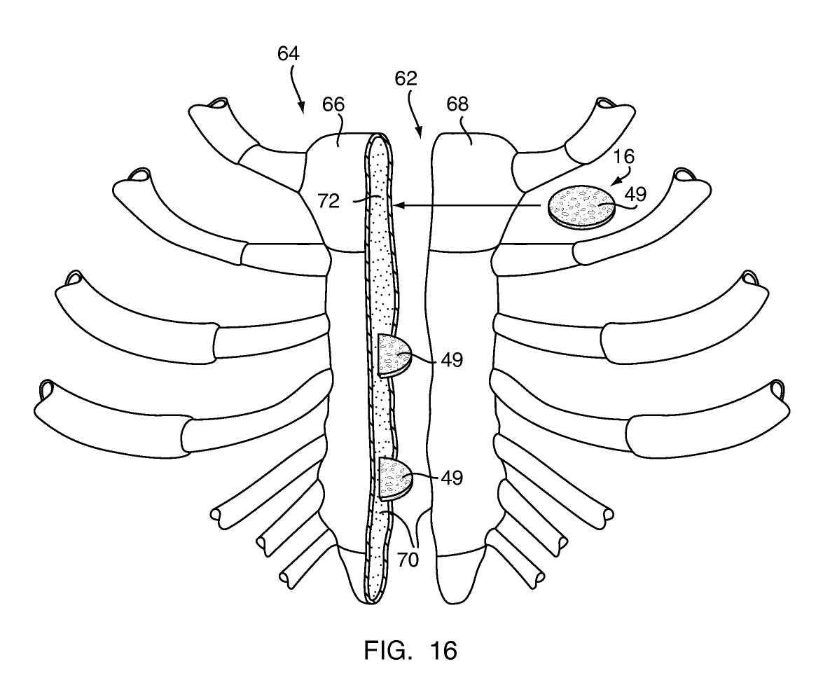

Referring to FIG. 16, in another embodiment, the biocompatible adhesive 16 shaped as disks 49 may advantageously be implemented for sternotomy repair. In this embodiment, the incision 62 is made in the sternum 64 to separate the sternum 64 into at least the first sternum portion 66 and the second sternum portion 68 and to expose the cancellous bone 72 at the cut surfaces 70 in the same manner discussed above. Then, disks 49 biocompatible adhesive 16 in suspended polymerization may be pressed into the cancellous bone 72 through one of the cut surfaces 70. Preferably, the disks 49 are pressed approximately halfway into the cancellous bone 72. Then, wires 74, shown in FIG. 15, or similar closure devices, are tightened to approximate the first and second sternum portions 66 and 68, forcing the exposed portion of each disk 49 into the cancellous bone 72 of the opposing sternum portion 66 or 68. Subsequent heating of the disks 49, for example, from the patient's body heat or another heating device accelerates polymerization and initiates expansion of the biocompatible adhesive 16 into the cancellous bone 72. This embodiment advantageously provides for expansion of the biocompatible adhesive 16 into the first and second sternum portions 66 and 68 through a mess free application relative to application of the biocompatible adhesive 16 in the liquid state 30, shown in FIG. 4, or the taffy state 32, shown in FIG. 4, using a spatula (not shown) or syringe (not shown).

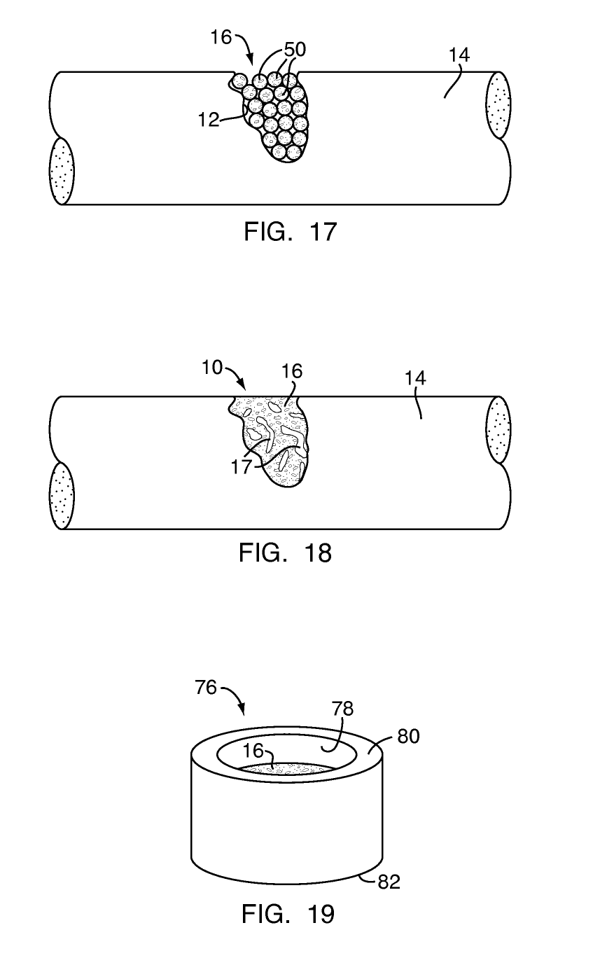

Referring to FIG. 17, biocompatible adhesive 16 in the form of granules 50, may be particularly advantageous for repair of irregular internal bone defects 12 or the like. In particular, a plurality of frozen granules 50 may be packed into the bone defect 12 in step S16, shown in FIG. 2. In some embodiments, the granules 50 may advantageously be combined with autograft, allograft or demineralized bone matrix to improve bone ingrowth after implantation. Once implanted, polymerization of the frozen granules 50 may be accelerated in step S18, shown in FIG. 2, as discussed above.

Referring to FIG. 18, as the granules 50, shown in FIG. 17, fully polymerize, the biocompatible adhesive 16 from the various granules 50, shown in FIG. 17, thaws and combines to provide the fully cured biocompatible adhesive 16 with a single body structure having large pores 17 formed from the gaps between the granules 50, shown in FIG. 17, and carbon dioxide byproducts of the polymerization process. This embodiment advantageously increases porosity of the fully cured bone defect filler 10 as well as the interconnectivity of the pores 17 within the bone defect filler 10, which promotes osteoconduction within the bone defect filler 10. This embodiment may also advantageously reduce expansion of the biocompatible adhesive 16 out of the bone defect 12, since some expansion of the biocompatible adhesive 16 will occupy the space between the granules 50, shown in FIG. 17.

Referring to FIG. 19, in yet another embodiment of the present invention, the premixed and frozen biocompatible adhesive 16 may be packaged within an interbody spacer 76 having an open inner core 78 extending from a superior surface 80 to an inferior surface 82. The premixed and frozen biocompatible adhesive 16 is packaged within the open inner core 78 in steps S10 through S14, shown in FIG. 2. Such interbody spacers 76 take variety of shapes and sizes and are used for interbody fusion procedures of the cervical, thoracic and lumbar spine. The interbody spacer 76 may be formed from conventional materials including PEEK, titanium or allograft bone. Alternately, the interbody spacer 76 may be formed of a polymer chemically similar to the biocompatible adhesive 16. In some embodiments, prior to freezing the biocompatible adhesive 16, the biocompatible adhesive 16 may be premixed with a bone graft (e.g. demineralized bone matrix) or with fully polymerized granules (not shown) of the biocompatible adhesive 16.

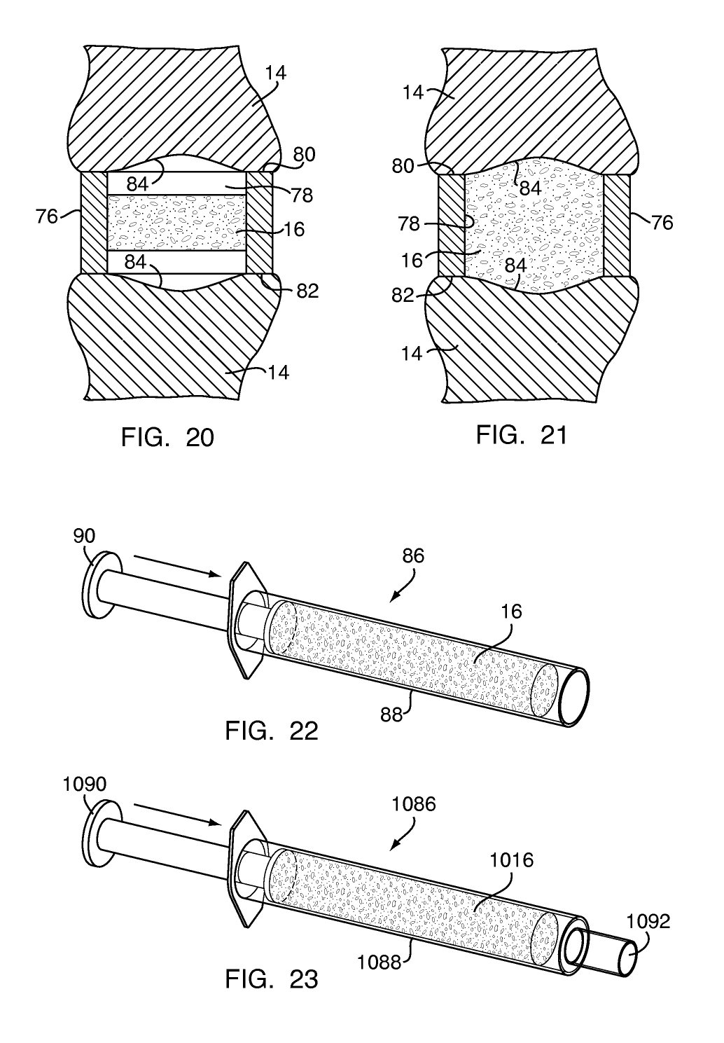

Referring to FIG. 20, when implanted in situ during interbody fusion in step S16, shown in FIG. 2, the superior end 80 and the inferior end 82 of the interbody spacer 76 are in direct communication with bony endplates 84 of bone 14. The biocompatible adhesive 16 begins to thaw under exposure to the patient's body temperature causing polymerization of the biocompatible adhesive 16 to accelerate at step S18, shown in FIG. 2.

Referring to FIG. 21, as the biocompatible adhesive 16 polymerizes, it expands, achieving direct contact with the bony endplates 84. The expanding biocompatible adhesive 16 may additionally chemically bond to or interdigitate with the bone 14 to achieve a superior implant stability relative to interbody spacers without biocompatible adhesive 16. Although this embodiment has been described in connection with an interbody fusion procedure, it may also advantageously be applicable for treating segmental bone defects of long bones or for serving as spacers for facet fusions.

Referring to FIG. 22, in another embodiment of the present invention, a bone filler device 86 is provided for packaging the biocompatible adhesive 16. The bone filler device 86 includes a long cannula 88 having a pusher 90 slidably engaged therein. The biocompatible adhesive 16 may be mixed, frozen, packaged, sterilized within the cannula 88 and transported to the hospital in the bone filler device 86 in steps S10 through S14, shown in FIG. 2.

Referring to FIG. 23, wherein like numerals represent like elements, in some embodiments, the bone filler device 1086 may be a small volume syringe, e.g. a syringe with a volume between one and two cubic centimeters (1-2 cc). The bone filler device 1086 includes a cannula 1088 with a pusher 1090 slidably engaged therein and a bore 1092 disposed at an opposite end of the cannula 1088 from the pusher 1090. The bore 1092 allows contents of the bone filler device 1086 to be expelled therethrough. Small volume syringes are advantageous because the small diameter of the cannula 1088 relative to the size of the bore 1092 provides excellent mechanical advantage, i.e. internal pressure, for expelling the biocompatible adhesive 1016 while in a viscous state. The small volume syringe is also advantageous since the small volume of biocompatible adhesive 1016 contained therein warms uniformly, whereas a larger volume may heat up at the edges but may remain frozen in the center.

Referring back to FIG. 22, at step S16, shown in FIG. 2, the bone filler device 86 is transferred to the operating room at the appropriate time in the surgical procedure. The biocompatible adhesive 16 disposed within the cannula 88 of the bone filler device 86 may then be sufficiently thawed and dispensed into the target site, e.g. bone defect 12, shown in FIG. 1, by driving the pusher 90 through the cannula 88. For example, the biocompatible adhesive 16 may be thawed in a hot water bath, within a sleeve containing an exothermic chemical reaction, in an electric heater or the like. Due to the length of the cannula 88, the bone filler device 86 is particularly useful in surgical procedures where the biocompatible adhesive 16 must be applied deep within the patient's tissue with limited exposure (e.g. vertebroplasty, MIS pedicle screw augmentation, percutaneous rib fracture, femoral neck fracture repair). In some embodiments of the bone filler device 86, the length and diameter of the cannula 88 may be increased and/or decreased to ease movement of the pusher 90 and, therefore, dispensing of the biocompatible adhesive 16 and to reduce compression of the biocompatible adhesive 16 during delivery, thereby maintaining the porous structure of the biocompatible adhesive 16. Additionally, in some embodiments, the pusher 90 may be configured with a threaded advance feature such that turning the pusher 90 causes it to move within the cannula 88.

Referring to FIG. 23, small volume syringes may also be advantageous for filling stripped screw holes. For example, in some embodiments, multiple bone filler devices 1086 may be packaged as a kit, e.g. for augmenting bone screws during a spine procedure. The bone filler devices 1086 may be moved to the operating room together and maintained on/in a cooling device (e.g. a cold metal plate with holes to accept the syringes). The surgeon may then use the syringes as needed during the procedure, for example, in five to ten minute increments, allowing polymerization of the biocompatible adhesive 16 within each syringe to accelerate as necessary.

Accordingly, the methods and devices of the present invention provide for improved handling and application of an in situ curing bone defect filler 10. Additionally, the method for forming and delivering the biocompatible adhesive 16 to the bone defect 12 advantageously provides the surgeon with bone defect filler 10 having a malleable osteoconductive structure without compromising the bone defect filler's physical and adhesive characteristics.

Additionally, the method according to the present invention advantageously decreases the susceptibility of the biocompatible adhesive 16 to contamination or the like during handling, transport and implantation thereof.

The method according to the present invention also advantageously eases implantation of the biocompatible adhesive 16 and reduces the likelihood of injury and harm to sensitive structures and organs proximate to the implantation site.

The method according to the present invention also advantageously reduces the operating room time required for the surgeon to mix and/or shape the biocompatible adhesive 16. Furthermore, the present invention advantageously allows the biocompatible adhesive 16 to be stored in various shapes, sizes and at various target viscosity points 38 during polymerization, thereby allowing the surgeon to select the best bone defect filler 10 for an intended application.

Although this invention has been shown and described with respect to the detailed embodiments thereof, it will be understood by those skilled in the art that various changes in form and detail thereof may be made without departing from the spirit and the scope of the invention. For example, although the present invention has been described as including a biocompatible adhesive 16, shown in FIG. 3, formed from the combination of multiple components, alternative adhesive formulations may be designed as one part systems where polymerization of the adhesive is triggered by moisture in the air or through the use of a chemically blocked isocyanate that requires heat to unblock the isocyanate to initiate polymerization.

What is claimed is:

1. A method for repairing a bone defect comprising:
preparing a biocompatible adhesive;
substantially suspending polymerization of the biocompatible adhesive prior to full cure;
delivering the biocompatible adhesive to the bone defect while polymerization is substantially suspended; and
allowing polymerization of the biocompatible adhesive to accelerate until full cure.

2. The method according to claim 1, wherein polymerization of the biocompatible adhesive is substantially suspended by freezing the biocompatible adhesive.

3. The method according to claim 1, wherein the biocompatible adhesive is prepared by mixing a prepolymer component, a polyol component and a filler material.

4. The method according to claim 1, wherein the biocompatible adhesive is a polyurethane adhesive.

5. The method according to claim 1, additionally comprising packaging the biocompatible adhesive to inhibit direct contact therewith.

6. The method according to claim 5, wherein the packaging includes at least one low surface energy sheet.

7. The method according to claim 1, additionally comprising transferring the biocompatible adhesive from a manufacturing facility to an operating facility while polymerization is substantially suspended.

8. The method according to claim 1, additionally comprising shaping the biocompatible adhesive into a shape selected from the group consisting of a rod, a sheet, a disk, a block and a ball.

9. The method according to claim 1, wherein the biocompatible adhesive is delivered to the bone defect with surgical gloves without the biocompatible adhesive substantially adhering to the surgical gloves.

10. The method according to claim 1, wherein polymerization of the biocompatible adhesive is substantially suspended before the biocompatible adhesive transitions out of a substantially adhesive state.

11. The method according to claim 1, wherein the biocompatible adhesive expands when polymerization accelerates.

12. The method according to claim 1, wherein polymerization of the biocompatible adhesive is substantially suspended after the biocompatible adhesive has undergone at least some expansion.

13. A bone repair apparatus comprising:
a partially polymerized biocompatible adhesive provided in a state wherein polymerization of the biocompatible adhesive is substantially halted.

14. The bone repair apparatus according to claim 13, wherein the temperature of the biocompatible adhesive is lowered to substantially halt polymerization of the biocompatible adhesive.

15. The bone repair apparatus according to claim 14, wherein the temperature of the biocompatible adhesive is lowered to below −20° C.

16. The bone repair apparatus according to claim 14, wherein polymerization of the biocompatible adhesive will accelerate as its temperature is elevated.

17. The bone repair apparatus according to claim 13, wherein the biocompatible adhesive has a shape selected from the group consisting of a rod, a sheet, a disk, a block and a ball.

18. The bone repair apparatus according to claim 13, wherein the biocompatible adhesive is packaged in a cylindrical tube.

19. The bone repair apparatus according to claim 18, wherein the cylindrical tube is a cannula of a syringe.

20. The bone repair apparatus according to claim 13, wherein the biocompatible adhesive is packaged in a low surface energy container.

21. The bone repair apparatus according to claim 13, wherein the biocompatible adhesive is packaged on a flexible container.

22. A bone repair product formed by a process comprising:
combining a prepolymer component, a polyol component and a filler material to form a mixture;
allowing the mixture to partially polymerize;
cooling the mixture to substantially halt polymerization thereof;
warming the mixture to accelerate polymerization thereof;
implanting the mixture in a surgical site; and
allowing the mixture to fully polymerize after implantation in the surgical site.

23. The bone repair product of claim 22, wherein the mixture is partially polymerized to a target viscosity point prior to substantially halting polymerization thereof.

24. The bone repair product of claim 23, wherein the target viscosity point occurs between four minutes and six minutes from initially combining the prepolymer component, polyol component and filler material.

25. The bone repair product of claim 22, wherein the mixture is a biocompatible polyurethane.

26. The bone repair product of claim 22, wherein the mixture is cooled to less then −20° C.

27. The bone repair product of claim 22, wherein the mixture is warmed to room temperature.

28. The bone repair product of claim 22, wherein the mixture is warmed to body temperature.

29. The bone repair product of claim 22, additionally formed by shaping the mixture prior to cooling.

30. The bone repair product of claim 29, wherein the mixture is shaped into a shape selected from the group consisting of a rod, a sheet, a disk, a block and a ball.

31. The bone repair product of claim 29, wherein the mixture is shaped by molding prior to cooling.

32. The bone repair product of claim 22, additionally formed by shaping the mixture by machining after cooling of the mixture.
